(19) United States
(12) Patent Application Publication
(10) Pub. No.: US 2022/0322998 A1
(43) Pub. Date: Oct. 13, 2022

(54) SLEEP STATE SENSING METHOD AND SLEEP STATE SENSING SYSTEM

(71) Applicant: Osense Technology Co., Ltd., Taipei City (TW)

(72) Inventors: You-Kwang Wang, Taipei City (TW); Zhi-Wei Wang, New Taipei City (TW)

(73) Assignee: Osense Technology Co., Ltd., Taipei City (TW)

(21) Appl. No.: 17/362,978

(22) Filed: Jun. 29, 2021

(30) Foreign Application Priority Data
Apr. 9, 2021 (TW) 110112832

(51) Int. Cl. A61B 5/00; A61B 5/024; A61B 5/11; A61B 5/08

(52) U.S. Cl. CPC A61B 5/4809; A61B 5/02405; A61B 5/7235; A61B 5/11; A61B 5/08; A61B 2560/0223; A61B 2562/0219; A61B 2562/0204

(57) ABSTRACT

A sleep state sensing method and a sleep state sensing system are provided. The sleep state sensing method includes: sensing a designated physiological state through an electronic device to generate a first physiological signal group and sensing the designated physiological state through a physiological signal sensing instrument to generate a second physiological signal group; calibrating the electronic device based on the first physiological signal group and the second physiological signal group such that the electronic device generates a third physiological signal group; and determining a sleep state through the third physiological signal group.

### CROSS-REFERENCE TO RELATED APPLICATION

This application claims the priority benefit of Taiwan application serial no. 110112832, filed on Apr. 9, 2021. The entirety of the above-mentioned patent application is hereby incorporated by reference herein and made a part of this specification.

### BACKGROUND

### Technical Field

The disclosure relates to a sleep state sensing method and a sleep state sensing system, and in particular, relates to a sleep state sensing method and a sleep state sensing system capable of improving sensing accuracy and sensing agreement.

### Description of Related Art

The quality of sleep is correlated with fatigue, and it also plays a key role in the recovery of physical, intellectual, and mental states. At present, the sleep state may be sensed by electronic devices (e.g., wearable electronic devices). Nevertheless, the electronic devices on the market are produced by different manufactures, and thus, the models and sensing mechanisms of these electronic devices are different, leading to different sensing accuracy and agreement as a result. Therefore, how to improve sensing accuracy and sensing agreement of electronic devices is an important issue.

### SUMMARY

The disclosure provides a sleep state sensing method capable of improving sensing accuracy and sensing agreement of an electronic device for a sleep state.

The disclosure provides a sleep state sensing method including the following steps. A designated physiological state is sensed through an electronic device to generate a first physiological signal group, and the designated physiological state is sensed through a physiological signal sensing instrument to generate a second physiological signal group. The electronic device is calibrated based on the first physiological signal group and the second physiological signal group, such that the electronic device modifies the first physiological signal group to a third physiological signal group. A sleep state is determined through the third physiological signal group.

The disclosure further provides a sleep state sensing system including an electronic device, a physiological signal sensing instrument, and an operator. The electronic device is configured to sense a designated physiological state to generate a first physiological signal group. The physiological signal sensing instrument is configured to sense the designated physiological state to generate a second physiological signal group. The operator is configured to communicate with the electronic device and the physiological signal sensing instrument and calibrate the electronic device based on the first physiological signal group and the second physiological signal group, such that the electronic device modifies the first physiological signal group to a third physiological signal group. A sleep state is determined by the electronic device through the third physiological signal group.

To sum up, in the sleep state sensing method and the sleep state sensing system provided by the disclosure, the electronic device is calibrated based on the second physiological signal group generated by the physiological signal sensing instrument. Therefore, high sensing agreement is provided between the calibrated electronic device and the physiological signal sensing instrument. In this way, in the disclosure, the sensing accuracy and sensing agreement of the electronic device for the sleep state may be improved.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 is a schematic diagram illustrating a sleep state sensing system according to an embodiment of the disclosure.

FIG. 2 is a method flow chart illustrating a sleep state sensing method according to a first embodiment of the disclosure.

FIG. 3 is a method flow chart illustrating the sleep state sensing method according to a second embodiment of the disclosure.

FIG. 4 is a method flow chart which is illustrated according to step S130.

FIG. 5 is a method flow chart which is illustrated according to step S134.

### DESCRIPTION OF THE EMBODIMENTS

Several embodiments of the disclosure are described in detail below accompanying with figures. In terms of the reference numerals used in the following descriptions, the same reference numerals in different figures should be considered as the same or the like elements. The embodiments are only a portion of the disclosure, which do not present all embodiments of the disclosure. More specifically, the embodiments serve as examples of the apparatus and method fall within the scope of the claims of the disclosure.

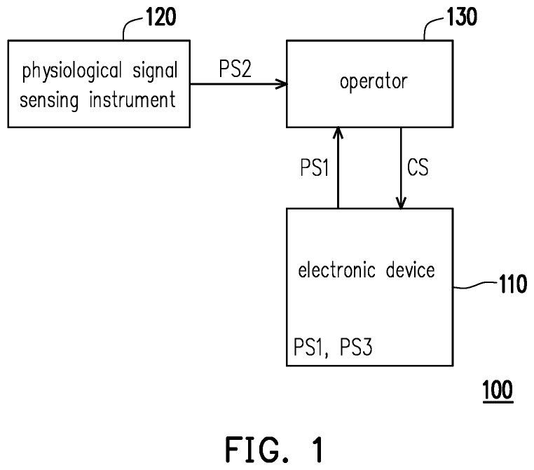

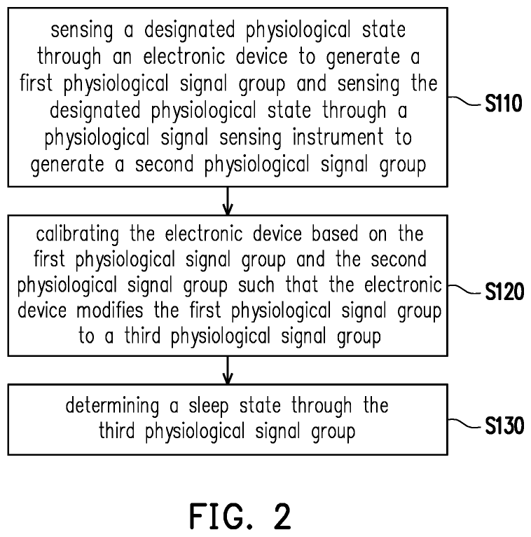

With reference to FIG. 1 and FIG. 2 together, FIG. 1 is a schematic diagram illustrating a sleep state sensing system according to an embodiment of the disclosure. FIG. 2 is a method flow chart illustrating a sleep state sensing method according to a first embodiment of the disclosure. In this embodiment, a sleep state sensing system 100 includes an electronic device 110, a physiological signal sensing instrument 120, and an operator 130. In this embodiment, the electronic device 110 senses a designated physiological state to generate a first physiological signal group PS1 in step S110. The electronic device 110 may be, for example, a wearable device, such as a smart watch, a smart bracelet, etc. In this embodiment, the physiological signal sensing instrument 120 senses the designated physiological state to generate a second physiological signal group PS2 in step S110. In this embodiment, the electronic device 110 and the physiological signal sensing instrument 120 sense the same designated physiological state. For instance, the designated physiological state may be a physiological state of a test sample (e.g., a subject or a user of the electronic device 110), such as a relaxed state or a sleep state. In this embodiment, the physiological signal sensing instrument 120 is a professional instrument (e.g., a BIOPAC series instrument or an Actigraph series instrument) for measuring human physiological signals.

In this embodiment, the operator 130 performs wired communication or wireless communication with the electronic device 110 and the physiological signal sensing instrument 120. The operator 130 calibrates the electronic device 110 based on the first physiological signal group PS1 and the second physiological signal group PS2 in step S120. In this embodiment, the operator 130 calibrates the electronic device 110 through a control signal CS. The electronic device 110 may change a sensing parameter in the electronic device 110 according to the control signal CS to allow the electronic device 110 to modify the first physiological signal group PS1 to a third physiological signal group PS3 (i.e., a modified first physiological signal group PS1).

In this embodiment, the operator 130 may be an apparatus or a device coupled to the electronic device 110 and the physiological signal sensing instrument 120. The operator 130 may be, for example, a personal computer, a notebook computer, a server, a database, a tablet computer, a smart electronic device, and other apparatuses and devices with computing capabilities.

In some embodiments, the operator 130 may be disposed inside the electronic device 110. The operator 130 may be, for example, a central processing unit (CPU), a programmable microprocessor for general or special use, a digital signal processor (DSP), a programmable controller, an application specific integrated circuit (ASIC), a programmable logic device (PLD), or any other similar devices or a combination of the foregoing devices, and may be loaded to run a computer program.

In this embodiment, in step S130, the electronic device 110 determines the sleep state (e.g., rapid eye movement (REM), non-rapid eye movement (NREM), NREM sleep has 3 stages, namely Stage N1: awakened easily, Stage N2: a small amount of slow waves (DELTA) brain sleep, and Stage N3: a large number of slow waves (Delta) brain wave sleep, and wake awake) of the user according to the third physiological signal group PS3.

Note that the operator 130 calibrates the electronic device 110 based on the second physiological signal group PS2 generated by the physiological signal sensing instrument 120 and the first physiological signal group PS1 generated by the electronic device 110. Therefore, high sensing agreement and sensing accuracy similar to that provided by the physiological signal sensing instrument 120 are provided between the calibrated electronic device 110 and the physiological signal sensing instrument 120. In this way, the sensing accuracy and sensing agreement of the electronic device 110 for the sleep state may be improved.

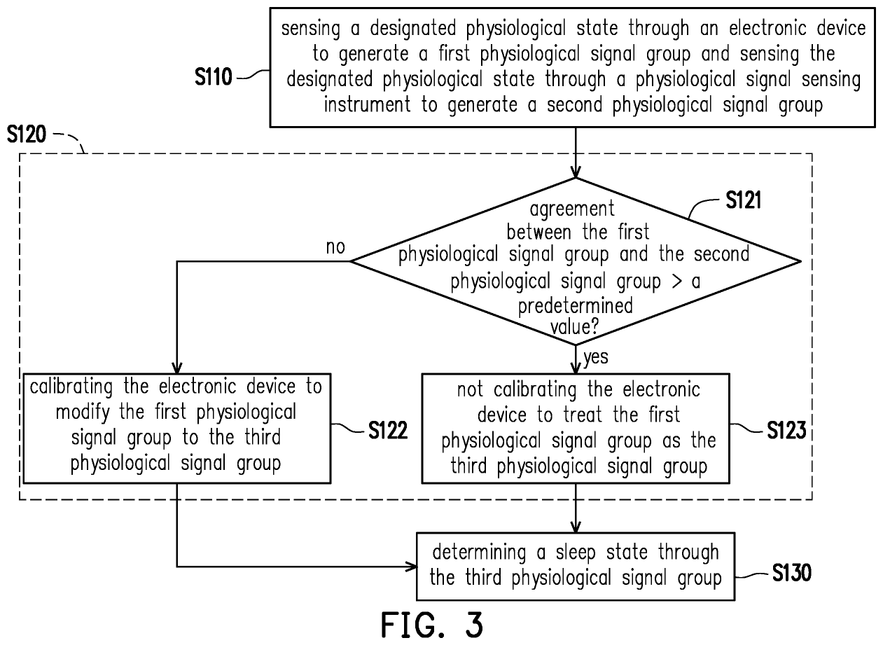

Several examples are provided as follows to describe the implementation details in step S120 and step S130. With reference to FIG. 1 and FIG. 3 together, FIG. 3 is a method flow chart illustrating the sleep state sensing method according to a second embodiment of the disclosure. The sleep state sensing method provided by this embodiment may be applied to the sleep state sensing system 100. In step S110, the electronic device 110 senses the designated physiological state to generate the first physiological signal group PS1. Similarly, in step S110, the physiological signal sensing instrument 120 senses the same designated physiological state to generate the second physiological signal group PS2. In this embodiment, the physiological signal sensing instrument 120 may be a BIOPAC series instrument. In this embodiment, step S120 includes steps S121 to S123. In step S121, the first physiological signal group PS1 and the second physiological signal group PS2 are analyzed to obtain agreement between the first physiological signal group PS1 and the second physiological signal group PS2. When the agreement between the first physiological signal group PS1 and the second physiological signal group PS2 is less than or equal to a predetermined value, the electronic device 110 is calibrated in step S122 to modify the first physiological signal group PS1 to the third physiological signal group PS3 and perform the operation of step S130. In this embodiment, agreement between the third physiological signal group PS3 and the second physiological signal group PS2 may be greater than the predetermined value. In contrast, when the agreement between the first physiological signal group PS1 and the second physiological signal group PS2 is greater than the predetermined value, the electronic device 110 directly treats the first physiological signal group PS1 as the third physiological signal group PS3 in step S123 and performs the operation of step S130. That is, the electronic device 110 is not required to be calibrated.

In this embodiment, the electronic device 110 may be a wearable device such as a smart bracelet or a smart watch. The first physiological signal group PS1 received by the electronic device 110 may include a heart rate signal of the subject. The second physiological signal group PS2 received by the physiological signal sensing instrument 120 may include the heart rate signal of the subject. The first physiological signal group PS1 and the second physiological signal group PS2 are analyzed to obtain the agreement between the first physiological signal group PS1 and the second physiological signal group PS2.

The operator 130 may obtain the heart rate signal, such as a vasoconstriction volume ratio (P-P interval, PPI) heart rate signal and the like, in the first physiological signal group PS1. The operator 130 may obtain the heart rate signal, such as a heartbeat waveform, in the second physiological signal group PS2. The operator 130 may perform a linear regression analysis based on the heart rate signal corresponding to the first physiological signal group PS1 and the heart rate signal corresponding to the second physiological signal group PS2, so as to accordingly obtain the agreement between the first physiological signal group PS1 and the second physiological signal group PS2. In some embodiments, the operator 130 may compare a correlation between the heart rate signal corresponding to the first physiological signal group PS1 and the heart rate signal corresponding to the second physiological signal group PS2 based on artificial intelligence to obtain the agreement between the first physiological signal group PS1 and the second physiological signal group PS2.

Incidentally, the heart rate signal (e.g., the vasoconstriction volume ratio) provided by the electronic device 100 may be different from the heart rate signal (e.g., the heartbeat waveform) provided by the physiological signal sensing instrument 120. Therefore, signal sampling restriction of the operator 130 may be significantly relaxed. That is, the disclosure may be applied to compare configurations of different heart rate signals to obtain the agreement between the first physiological signal group PS1 and the second physiological signal group PS2.

In this embodiment, the operator 130 determine the agreement between the first physiological signal group PS1 and the second physiological signal group PS2 in step S121. The designated physiological state is the same, and the second physiological signal group PS2 is an accurate physiological signal. As such, when the agreement is less than or equal to the predetermined value (e.g., 95%, which is not limited by the disclosure), it means that the first physiological signal group PS1 is not accurate. Therefore, the operator 130 calibrates the electronic device 110 in step S122 until the agreement between the modified first physiological signal group PS1 provided by the electronic device 110 and the second physiological signal group PS2 is greater than the predetermined value.

The predetermined value provided by this embodiment may be modified according to specification needs. It should be understood that when the predetermined value increases, the third physiological signal group PS3 is closer to the second physiological signal group PS2. Nevertheless, the number of times of calibration increases as well.

Note that once the third physiological signal group PS3 of the electronic device 110 is generated, operations of steps S110 and S120 are not required to be performed.

In step S130, the electronic device 110 determines the sleep state of the user according to the third physiological signal group PS3. In this embodiment, the electronic device 110 analyzes the third physiological signal group PS3 to obtain a pulse rate variability (PRV) message and determines whether the pulse rate variability message corresponding to the third physiological signal group PS3 meets one of pulse rate variability indicators corresponding to the above-mentioned multiple sleep states. For instance, the electronic device 110 may perform at least one of a time-domain analysis and a frequency-domain analysis on the third physiological signal group PS3 to obtain the pulse rate variability message.

Regarding the analysis based on the time-domain analysis, the pulse rate variability message may be at least one of a mean of all normal heart rates (MeanHR), a mean of all normal to normal intervals (MeanRR), a standard deviation of all normal heart rates (SDHR), a standard deviation of all normal to normal intervals (SDNN), a root mean square of successive differences between normal to normal interval (R_MSSD), a number of pairs of successive NNs that differ by more than 50 ms (NN50), and a proportion of NN50 divided by total number of NNs (pNN50).

Further, regarding the analysis based on the frequency-domain analysis, the pulse rate variability message may be at least one of a sum of heartbeat variability within a frequency spectrum range ≤0.4 Hz (frequency spectrum range ≤0.4 Hz), a variation of the normal heartbeat interval in a high frequency range (frequency spectrum range is 0.15 to 0.4 Hz), a variation of the normal heartbeat interval in a low frequency range (frequency spectrum range is 0.04 to 0.15 Hz), a variation of the normal heartbeat interval in an excessively low frequency range (frequency spectrum range is 0.003 to 0.04 Hz), a quantitative indicator of parasympathetic nerve activity, a quantitative indicator of sympathetic nerve activity, and an indicator of sympathetic and parasympathetic balance.

In step S130, the electronic device 110 determines whether the pulse rate variability message meets one of the pulse rate variability indicators corresponding to the multiple sleep states. For instance, when it is determined that the pulse rate variability message meets the pulse rate variability indicator corresponding to the light sleep state, the electronic device 110 may determine that the user is in the light sleep state.

In an embodiment, the first physiological signal group PS1 may include a movement signal of a body movement of the subject. The second physiological signal group PS2 may include the movement signal of the body movement of the subject. The electronic device 110 may include a movement sensor (e.g., an accelerometer or a gyroscope). In step S110, the electronic device 110 may sense and treat at least one of an acceleration change of the body movement of the subject, a length of time during which the body movement changes (that is, the length of time that the body movement continues to change), and a length of time that the body movement stops as the movement signal through the movement sensor. The electronic device 110 may initialize the movement sensor by standing still before performing sensing. In step S110, the physiological signal sensing instrument 120 may sense and treat at least one of an acceleration change of the same body movement of the subject, a length of time during which the body movement changes, and a length of time that the body movement stops as the movement signal. In this embodiment, the physiological signal sensing instrument 120 may be an Actigraph series instrument.

In step S121, the operator 130 obtains a first plurality of acceleration components of the first physiological signal group PS1 in a three-dimensional space in different directions and obtains a second plurality of acceleration components of the second physiological signal group PS2 in the three-dimensional space in different directions. Next, the operator 130 calibrates the electronic device 110 based on the first plurality of acceleration components and the second plurality of acceleration components. In this embodiment, the operator 130 compares between the first plurality of acceleration components and the second plurality of acceleration components to obtain agreement between the first plurality of acceleration components and the second plurality of acceleration components. When the agreement between the first plurality of acceleration components and the second plurality of acceleration components is less than or equal to the predetermined value, the operator 130 calibrates the electronic device 110 in step S122. In contrast, when the agreement between the first plurality of acceleration components and the second plurality of acceleration components is greater than the predetermined value, the operator 130 does not calibrate the electronic device 110 (e.g., the operation in step S123).

In this embodiment, generally, different sleep states have different turnover characteristic indicators. That is, different sleep states correspond to different turnover movements, such as a turnover range and a turnover frequency. In step S130, the electronic device 110 analyzes the third physiological signal group PS3 to obtain the movement message and determines whether the movement message meets one of the turnover characteristic indicators. For instance, when it is determined that the movement message meets the turnover characteristic indicator corresponding to the deep sleep state, the electronic device 110 may determine that the user is in the deep sleep state.

In an embodiment, the first physiological signal group PS1 may include a breathing message of the subject. The second physiological signal group PS2 may also include the breathing message of the subject. The electronic device 110 may include a microphone module of any type. In step S110, the electronic device 110 may sense and treat a breathing sound of the subject as a first breathing audio signal through the microphone module. Similarly, the physiological signal sensing instrument 120 may also sense and treat the breathing sound of the subject as a second breathing audio signal. The physiological signal sensing instrument 120 may be a BIOPAC series instrument.

In this embodiment, the operator 130 compares between the first breathing audio signal and the second breathing audio signal to obtain agreement between the first breathing audio signal and the second breathing audio signal in step S121. For instance, the operator 130 may obtain a first frequency spectrogram corresponding to the first breathing audio signal and a second frequency spectrogram corresponding to the second breathing audio signal through a Fourier transformation manner in a specific interval. The operator 130 may obtain the agreement between the first breathing audio signal and the second breathing audio signal according to similarity between the first frequency spectrogram and the second frequency spectrogram.

In this embodiment, when the agreement between first breathing audio signal and the second breathing audio signal is less than or equal to the predetermined value, the operator 130 calibrates the electronic device 110 in step S122. In contrast, when the agreement between the first breathing audio signal and the second breathing audio signal is greater than the predetermined value, the operator 130 does not calibrate the electronic device 110 (e.g., the operation in step S123).

In this embodiment, different sleep states correspond to different sleep breathing behaviors, such as the speed of breathing and the frequency of breathing sounds. In step S130, the electronic device 110 analyzes the third physiological signal group PS3 to obtain a breathing message and determines whether the breathing message meets one of sleep breathing indicators. For instance, when it is determined that the movement message meets the sleep breathing indicator corresponding to the light sleep state, the electronic device 110 may determine that the user is in the light sleep state.

In some embodiments, the first physiological signal group PS1 includes at least two of the heart rate signal, the movement signal of the body movement, and the breathing audio signal. The second physiological signal group PS2 is required to include at least two of the heart rate signal, the movement signal of the body movement, and the breathing audio signal as well. Sufficient teachings regarding determination of the agreement and determination of the sleep state based on the heart rate signal, the movement signal, and the breathing audio signal may be obtained through the foregoing embodiments, and description thereof is not repeated herein.

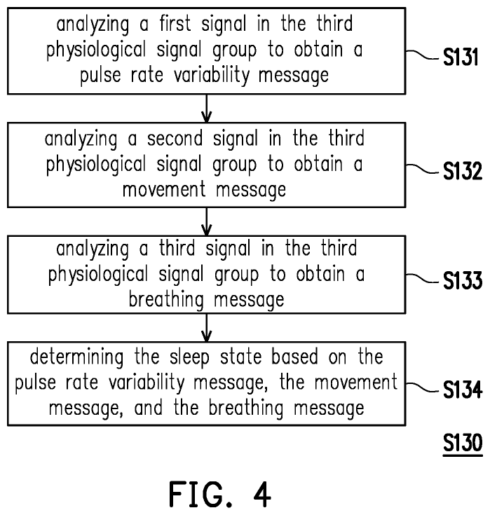

Examples regarding detailed implementation of determination of the sleep state based on the heart rate signal, the movement signal, and the breathing audio signal are provided as follows. With reference to FIG. 1 and FIG. 4 together, FIG. 4 is a method flow chart which is illustrated according to step S130. In this embodiment, the third physiological signal group PS3 at least includes a first signal, a second signal, and a third signal. The first signal, the second signal, and the third signal are all related to signals of different physiological indicators. In this embodiment, the first signal is the heart rate signal, the second signal is the movement signal, and the third signal is the breathing audio signal. Step S130 includes steps S131 to S134. In step S131, the electronic device 110 analyzes the first signal to obtain the pulse rate variability message. In step S132, the electronic device 110 analyzes the second signal to obtain the movement message. In step S133, the electronic device 110 analyzes the third signal to obtain the breathing message. In this embodiment, the order of steps S131 to S133 may be randomly changed. The order of steps S131 to S133 provided by the disclosure is not limited to the order provided by this embodiment.

In this embodiment, in step S134, the electronic device 110 may determine the sleep state based on the pulse rate variability message, the movement message, and the breathing message obtained in steps S131 to S133. In this embodiment, when the sleep states which are determined based on the pulse rate variability message, the movement message, and the breathing message refer to the same sleep indicator, the electronic device 110 may clearly determine the current sleep state of the user. In this embodiment, steps S131 to S134 may be performed through a processor in the electronic device 110, for example. The processor of the electronic device 110 may be, for example, a central processing unit (CPU), a programmable microprocessor for general or special use, a digital signal processor (DSP), a programmable controller, an application specific integrated circuit (ASIC), a programmable logic device (PLD), or any other similar devices or a combination of the foregoing devices, and may be loaded to run a computer program.

When the sleep states which are determined based on the pulse rate variability message, the movement message, and the breathing message refer to sleep indicators which are not exactly the same, the electronic device 110 may determine the current sleep state of the user based on a weight calculation method.

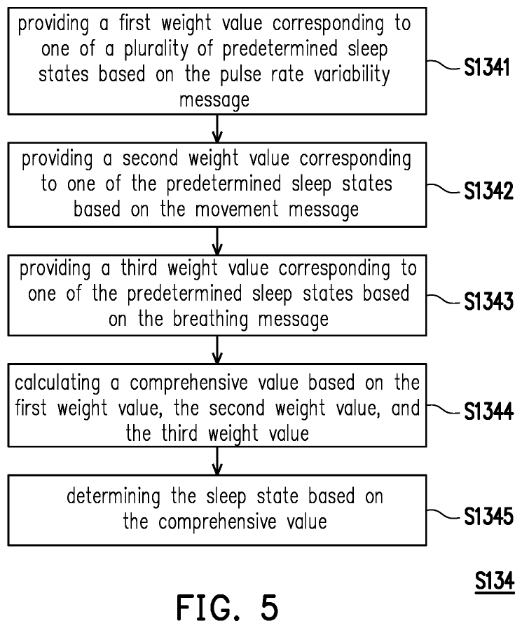

Examples of implementation of the weight calculation method are provided as follows. With reference to FIG. 1, FIG. 4, and FIG. 5 together, FIG. 5 is a method flow chart which is illustrated according to step S134. In this embodiment, step S134 includes steps S1341 to S1345. In step S1341, the electronic device 110 may provide a first weight value corresponding to one of a plurality of predetermined sleep states (e.g., the deep sleep state, light sleep state, quiescent sleep state, etc., which is not limited by the disclosure) based on the pulse rate variability message (i.e., the pulse rate variability message obtained in step S132). In step S1342, the electronic device 110 may provide a second weight value corresponding to one of the predetermined sleep states based on the movement message (i.e., the movement message obtained in step S132). In step S1343, the electronic device 110 may provide a third weight value corresponding to one of the predetermined sleep states based on the breathing message (i.e., the breathing message obtained in step S133).

In step S1344, the electronic device 110 may calculate a comprehensive value based on the first weight value, the second weight value, and the third weight value. In this embodiment, the electronic device 110 may calculate the comprehensive value corresponding to the first weight value, the second weight value, and the third weight value through a designed polynomial. In this embodiment, the polynomial may a ternary polynomial of any form which is obtained by, for example, linear regression. The first weight value, the second weight value, and the third weight value are regarded as three different variables. Therefore, in this embodiment, in step S1344, the electronic device 110 may substitute the first weight value, the second weight value, and the third weight value into the aforementioned polynomial to calculate the comprehensive value. In step S1345, the electronic device 110 may determine the sleep state according to the comprehensive value. In this embodiment, based on the polynomial, the comprehensive value is given the same physical meaning as the first weight value, the second weight value, and the third weight value. Therefore, is a result corresponding to one of the multiple predetermined sleep states. In this embodiment, the order of steps S1341 to S1345 may be randomly changed. The order of steps S1341 to S1345 provided by the disclosure is not limited to the order provided by this embodiment.

In some embodiments, based on the above weight calculation method, in steps S131 to S134, the electronic device 110 may integrate the first signal, the second signal, and the third signal of the third physiological signal group PS3 into a fourth signal (or called as a fourth physiological signal) including the comprehensive value. The electronic device 110 may determine the current sleep state of the user according to the comprehensive value of the fourth signal.

In view of the foregoing, in the sleep state sensing method and the sleep state sensing system provided by the disclosure, the electronic device is calibrated based on the second physiological signal group generated by the physiological signal sensing instrument. Therefore, high sensing agreement is provided between the calibrated electronic device and the physiological signal sensing instrument. In this way, in the disclosure, the sensing accuracy of the electronic device for the sleep state is improved, and sensing agreement is ensured among a plurality of electronic devices. In addition, the third physiological signal group includes the first signal, the second signal, and the third signal related to different physiological indicators. The electronic device may perform weight calculation to determine the current sleep state of the user based on the first signal, the second signal, and the third signal.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A sleep state sensing method, comprising:
sensing a designated physiological state through an electronic device to generate a first physiological signal group and sensing the designated physiological state through a physiological signal sensing instrument to generate a second physiological signal group;
calibrating the electronic device based on the first physiological signal group and the second physiological signal group such that the electronic device modifies the first physiological signal group to a third physiological signal group; and
determining a sleep state through the third physiological signal group.

2. The sleep state sensing method according to claim 1, wherein the step of calibrating the electronic device based on the first physiological signal group and the second physiological signal group comprises:
analyzing the first physiological signal group and the second physiological signal group to obtain agreement between the first physiological signal group and the second physiological signal group; and
calibrating the electronic device when the agreement between the first physiological signal group and the second physiological signal group is less than a predetermined value.

3. The sleep state sensing method according to claim 1, wherein each of the first physiological signal group and the second physiological signal group comprises a heart rate signal and a pulse rate signal.

4. The sleep state sensing method according to claim 3, wherein the step of determining the sleep state through the third physiological signal group comprises:
analyzing one signal in the third physiological signal group to obtain a pulse rate variability message; and
determining whether the pulse rate variability message meets one of a plurality of pulse rate variability indicators.

5. The sleep state sensing method according to claim 4, wherein the step of analyzing one signal in the third physiological signal group to obtain the pulse rate variability message comprises:
performing at least one of a time-domain analysis and a frequency-domain analysis on one signal in the third physiological signal group to obtain the pulse rate variability message.

6. The sleep state sensing method according to claim 1, wherein each of the first physiological signal group and the second physiological signal group comprises a movement signal in response to a body movement.

7. The sleep state sensing method according to claim 6, wherein the step of sensing the designated physiological state through the electronic device to generate the first physiological signal group comprises:
sensing at least one of an acceleration change of the body movement, a length of time during which the body movement changes, and a length of time that the body movement stops through the electronic device.

8. The sleep state sensing method according to claim 6, wherein the step of calibrating the electronic device based on the first physiological signal group and the second physiological signal group comprises:
obtaining a first plurality of acceleration components of the first physiological signal group in a three-dimensional space in different directions;
obtaining a second plurality of acceleration components of the second physiological signal group in the three-dimensional space in different directions; and
calibrating the electronic device based on the first plurality of acceleration components and the second plurality of acceleration components.

9. The sleep state sensing method according to claim 7, wherein the step of determining the sleep state through the third physiological signal group comprises:
analyzing one signal in the third physiological signal group to obtain a movement message; and
determining whether the movement message meets one of a plurality of turnover characteristic indicators.

10. The sleep state sensing method according to claim 1, wherein each of the first physiological signal group and the second physiological signal group comprises a breathing audio signal.

11. The sleep state sensing method according to claim 10, wherein the step of determining the sleep state through the third physiological signal group comprises:
analyzing one signal in the third physiological signal group to obtain a breathing message; and
determining whether the breathing message meets one of a plurality of sleep breathing indicators.

12. The sleep state sensing method according to claim 1, wherein the step of determining the sleep state through the third physiological signal group comprises:
analyzing a first signal in the third physiological signal group to obtain a pulse rate variability message;
analyzing a second signal in the third physiological signal group to obtain a movement message in response a body movement;
analyzing a third signal in the third physiological signal group to obtain a breathing message; and
determining the sleep state based on the pulse rate variability message, the movement message, and the breathing message.

13. The sleep state sensing method according to claim 12, wherein the step of determining the sleep state based on the pulse rate variability message, the movement message, and the breathing message comprises:
providing a first weight value corresponding to one of a plurality of predetermined sleep states based on the pulse rate variability message;
providing a second weight value corresponding to one of the predetermined sleep states based on the movement message;
providing a third weight value corresponding to one of the predetermined sleep states based on the breathing message;
calculating a comprehensive value based on the first weight value, the second weight value, and the third weight value; and
determining the sleep state based on the comprehensive value.

14. A sleep state sensing system, comprising:
an electronic device, configured to sense a designated physiological state to generate a first physiological signal group;
a physiological signal sensing instrument, configured to sense the designated physiological state to generate a second physiological signal group; and
an operator, configured to communicate with the electronic device and the physiological signal sensing instrument and calibrate the electronic device based on the first physiological signal group and the second physiological signal group such that the electronic device modifies the first physiological signal group to a third physiological signal group,
wherein the electronic device determines a sleep state through the third physiological signal group.
